(19) United States
(12) Patent Application Publication
(10) Pub. No.: US 2010/0190734 A1
(43) Pub. Date: Jul. 29, 2010

(54) METHOD OF TREATING DRY EYE DISEASE WITH AZITHROMYCIN

(76) Inventors: Romulus Kimbro Brazzell, Morrisville, NC (US); Reza M. Haque, Randolph, NJ (US); Kurt E. Brubaker, Durham, NC (US); Robert J. Dempsey, Middleton, MA (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 12/691,575

(22) Filed: Jan. 21, 2010

Related U.S. Application Data
(60) Provisional application No. 61/147,013, filed on Jan. 23, 2009.

(51) Int. Cl. A61K 31/7052; A61P 27/02

(52) U.S. Cl. 514/29

(57) ABSTRACT

The present invention relates to a method for treating dry eye disease. The method comprises identifying a subject suffering from dry eye disease, and administering to the subject an amount of azithromycin effective to reduce dry eye symptoms and/or signs ands to improve tear film quality. Azithromycin is preferably administered topically to the subject in an aqueous ophthalmic solution comprises 0.5-1.5% (w/v) azithromycin in a polymeric suspension.

This application claims priority to U.S. Provisional Application No. 61/147,013, filed Jan. 23, 2009; the content of which is incorporated herein by reference in its entirety.

### TECHNICAL FIELD

This invention relates to a method of treating dry eye disease in a subject. The method is useful in relieving dry eye signs and symptoms. The method involves administering to the subject in need thereof azithromycin. The present invention is illustrated by administering to the subject an azithromycin ophthalmic solution, such as AZASITE®.

### BACKGROUND OF THE INVENTION

Dry eye disease is the general term for indications produced by abnormalities of the precorneal tear film characterized by a decrease in tear production or an increase in tear film evaporation, together with the ocular surface disease and symptoms that result. Approximately 38 million Americans are affected with some type of dry eye disorder. Among the indications that are referred to by the general term “dry eye disease” are: keratoconjunctivitis sicca (KCS), age-related dry eye, Stevens-Johnson syndrome, Sjogren's syndrome, ocular cicatrical pemphigoid, corneal injury, infection, Riley-Day syndrome, congenital alacrima, nutritional disorders or deficiencies (including vitamins), pharmacologic side effects, contact lens intolerance, eye stress and glandular and tissue destruction, environmental exposure to smog, smoke, excessively dry air, airborne particulates, autoimmune and other immunodeficient disorders, and comatose patients rendered unable to blink.

Dry eye disease, although seen pathologically during ophthalmic exams as superficial punctate keratopathy (SPK) of the ocular surface epithelium, is largely a symptomatic disease. Chronic dryness leads to pain and irritation that is often debilitating to the subject, preventing the performance of normal daily activities such as reading, driving, etc. Dry eye is most common in postmenopausal women; however, hormone replacement therapy has not been proven to help dry eye signs and symptoms.

Currently, the pharmaceutical treatment of dry eye disease is mostly limited to administration of artificial tears (saline solution) or anti-inflammatory agents (cyclosporine, steroids). Secretagogues (diquafosol, 15-HETE, rebamipide) to increase the production of tears are currently under development. In addition, artificial tears often have contraindications and incompatibility with soft contact lenses (M. Lemp, Cornea 9 (1), S48-550 (1990)). The use of phosphodiesterase inhibitors, such as 3-isobutyl-1-methylxanthine (IBMX) to stimulate tear secretion is disclosed in U.S. Pat. No. 4,753,945. The effectiveness of these phosphodiesterase inhibitors has been investigated (J. Gilbard, et al., Arch. Ophthal, 112, 1614-16 (1994) and 109, 672-76 (1991); idem, Inv. Ophthal. Vis. Sci. 31, 1381-88 (1990)). Stimulation of tear secretion by topical application of melanocyte stimulating hormones is described in U.S. Pat. No. 4,868,154. Although these interventions can reduce inflammation and/or reduce SPK associated with dry eye, they have not been proven to significantly reduce the symptoms of dry eye.

Dry eye disease is different from blepharitis; the two diseases have different patient populations. Dry eye symptoms are dryness, burning, photophobia, foreign body sensation and grittiness in the eyes. Dry eye disease is characterized by an insufficient or defective tear film. The primary end points for studying dry eye diseases are corneal and conjunctival staining, measuring tear volume (Schirmer tests), Tear break-up time, dryness, burning, photophobia, foreign body sensation and grittiness.

Blepharitis is a chronic disorder producing inflammation of the anterior and posterior eyelid margin, with involvement of skin and its related structures (hairs and sebaceous glands), the mucocutaneous junction, and the meibomian glands (American Academy of Ophthalmology Preferred Practice Pattern 2003; Thygeson 1946; Foulks 2003). Blepharitis has historically been treated on a chronic basis (Dougherty 1984) through either mechanical therapy (consisting of improved eyelid hygiene and eyelid compression) alone or in combination with topical or systemic antibiotics. Typical clinical signs of blepharitis include lid debris, redness of eyelid margin, eyelid swelling, plugging of the meibomian gland, and obstructed meibomian gland secretion. The primary endpoints for studying blepharitis include the amount of eyelid debris and hyperemia of the eyelid margin.

Azithromycin is a macrolide antibiotic. AZASITE® (azithromycin ophthalmic solution) is a 1% sterile aqueous topical ophthalmic solution of azithromycin formulated in DURASITE® (polycarbophil, edetate disodium, sodium chloride). AZASITE® is approved by the U.S. Food and Drug Administration (FDA) for treatment of bacterial conjunctivitis, caused by susceptible isolates of CDC coryneform group G, Haemophilus influenzae, Staphylococcus aureus, Streptococcus mitis group, and Streptococcus pneumoniae (AZASITE® Package Insert, 2008).

As a result of the ineffectiveness and inconvenience of current therapies of dry eyes, there remains a need for a method of treating dry eye disease, which is not only effective, but also free of significant side effects.

### SUMMARY OF THE INVENTION

The present invention is directed to a method of treating dry eye disease or reducing one or more dry eye signs and/or symptoms in a subject in need of such treatment. The method comprises the step of first identifying a subject suffering from dry eye disease or dry eye symptoms, then administering to the subject an effective amount of azithromycin.

Particularly, the present invention is suitable for treating dry eye diseases caused by one or more of keratoconjunctivitis sicca, age-related dry eye, Stevens-Johnson syndrome, Sjogren's syndrome, ocular cicatrical pemphigoid, corneal injury, infection, Riley-Day syndrome, congenital alacrima, nutritional disorders or deficiencies, pharmacologic side effects, contact lens intolerance, eye stress resulting in glandular and tissue destruction, autoimmune disorders, immuno-deficient disorders, comatose patients who are unable to blink, or environmental exposure to smog, smoke, excessively dry air, or airborne particulates. The present invention is also suitable to treat patients suffering from dry eye disease, but not suffering from blepharitis.

The present invention is also directed to a method for treating dry eye diseases in a subject prior to an ophthalmic surgical procedure. The method comprises the steps of: identifying a subject having dry eye disease or being at risk of developing post surgical dry eye disease, and administering to the subject an effective amount of azithromycin prior to the ophthalmic surgical procedure.

The present invention is further directed to a method for treating dry eye diseases in a subject following an ophthalmic surgical procedure. The method comprises the steps of: identifying a subject suffering from dry eye disease following an ophthalmic surgical procedure, and administering to the subject azithromycin.

Azithromycin is preferably administered topically to the subject in an aqueous ophthalmic solution comprises 0.5-1.5% (w/v) azithromycin in a polymeric suspension.

### DETAILED DESCRIPTION OF THE INVENTION

The present invention is directed to a method of treating dry eye disease or reducing dry eye signs and/or symptoms such as dryness, burning, photophobia, foreign body sensation and grittiness in a subject. The present invention is also directed to a method of improving the tear film in a subject suffering from dry eye symptoms. The method comprises the step of first identifying a subject suffering from dry eye disease or dry eye symptoms, then administering to the subject an effective amount of azithromycin. In one embodiment, azithromycin is administered in an aqueous ophthalmic solution comprising no additional active pharmaceutical ingredient.

The inventors have discovered that azithromycin reduces signs and symptoms of dry eye and improves the tear film in patients with dry eye disease. The method of the present invention is an improvement upon the current most commonly used treatment of dry eye disease—artificial tears (i.e., saline solution) and anti-inflammatory agents (cyclosporine). A normal tear film is composed of a mucin layer, an aqueous component and a lipid layer. The present method improves the quality of a patient's own tear film. The present method also provides topical analgesia of the symptomatic corneal irritation that occurs in dry eye.

Dry eye symptoms can be due to one or more of keratoconjunctivitis sicca (KCS), age-related dry eye, contact lens intolerance, Stevens-Johnson syndrome, Sjogren's syndrome, ocular cicatrical pemphigoid, corneal injury, infection, Riley-Day syndrome and congenital alacrima. Dry eye symptoms can also be caused by nutritional (such as vitamin) disorders or deficiencies, autoimmune disorders, immuno-deficient disorders, pharmacologic side effects, eye stress resulting in glandular and tissue destruction, and environmental exposure to smog, smoke, excessively dry air, or airborne particulates.

The present invention is also useful in reducing dry eye symptoms associated with contact lens wear in a subject who develops contact lens intolerance due to dryness in the eyes. The method of the present invention can enhance the number of hours of total contact lens wearing time, or can make wearing contact lens more comfortable to the user.

The present invention is useful as a wash or irrigation solution to eyes of those who are unable to blink, for example, patients who cannot blink due to muscle or nerve damage, neuromuscular blockade or loss of the eyelids, comatose patients, or conscious individuals during surgery.

The present invention is suitable for treating any dry eye diseases. Particularly, the present invention is suitable for treating dry eye diseases caused by one or more of keratoconjunctivitis sicca, age-related dry eye, Stevens-Johnson syndrome, Sjogren's syndrome, ocular cicatrical pemphigoid, corneal injury, infection, Riley-Day syndrome, congenital alacrima, nutritional disorders or deficiencies, pharmacologic side effects, contact lens intolerance, eye stress and glandular and tissue destruction, autoimmune disorders, immuno-deficient disorders, comatose patients who are unable to blink, or environmental exposure to smog, smoke, excessively dry air, or airborne particulates. The present invention is also suitable to treat patients suffering from dry eye disease, but not suffering from blepharitis. Subjects suitable for treatment by the present method often have had a clinical diagnosis of dry eye disease by a trained eye care professional.

The present method comprises the steps of: (i) identifying a subject suffering from dry eye disease; and (ii) administering to the eyes of the subject an aqueous ophthalmic solution comprising: (a) an active pharmaceutical ingredient consisting essentially of azithromycin, and (b) a physiologically compatible ophthalmic vehicle; wherein said dry eye disease is caused by one or more of keratoconjunctivitis sicca, age-related dry eye, Stevens-Johnson syndrome, Sjogren's syndrome, ocular cicatrical pemphigoid, corneal injury, infection, Riley-Day syndrome, congenital alacrima, nutritional disorders or deficiencies, pharmacologic side effects, contact lens intolerance, eye stress resulting in glandular and tissue destruction, autoimmune disorders, immuno-deficient disorders, comatose patients who are unable to blink, or environmental exposure to smog, smoke, excessively dry air, or airborne particulates.

In another embodiment, the present method comprises the steps of: (i) identifying a subject suffering from dry eye disease and not suffering from blepharitis; and (ii) administering to the subject an aqueous ophthalmic solution comprising: (a) an active ingredient consisting essentially of azithromycin, and (b) a physiologically compatible ophthalmic vehicle.

In another embodiment, the present method comprises the steps of: (i) identifying a subject suffering from dry eye disease; and (ii) administering to the eyes of the subject an aqueous ophthalmic solution comprising: (a) active pharmaceutical ingredients consisting essentially of azithromycin and a secretegogue, and (b) a physiologically compatible ophthalmic vehicle. A secretegogue is a compound or agent that induces stored material to be released from cells or tissue. Examples of secretegogues include diquafosol, 15-HETE, or rebamipide.

The present invention is also directed to a method for treating dry eye diseases in a subject prior to an ophthalmic surgical procedure. The method comprises the steps of: identifying a subject having dry eye disease or being at risk of developing post surgery dry eye disease, and administering to the subject an effective amount of azithromycin prior to an ophthalmic surgical procedure. Types of surgeries beneficial for the azithromycin pre-treatment include cataract surgery, refractive surgery such as PRK and Lasik, glaucoma surgery, corneal transplantation (keratoplasty), chalazion (acute or chronic eye lid lump) surgery, and pterygium (growth in cornea) surgery. The subject benefits from having his dry eye treated before surgery, because it improves his quality of life by reducing the symptoms, improving visual acuity, and reducing corneal epithelial defect.

Many patients develop dry eye symptoms following ophthalmic surgery. The present invention is further directed to a method for treating dry eye diseases in a subject following an ophthalmic surgical procedure. The method comprises the steps of: identifying a subject suffering from dry eye disease following an ophthalmic surgical procedure, and administering to the subject azithromycin.

After treatment by the present methods, one or more dry eye signs and/or symptoms are reduced or alleviated in the subject. Dry eye symptoms include dryness, burning, ocular itching, photophobia, foreign body sensation, and grittiness. Dry eye signs are assessed by measurements such as: corneal and/or conjunctival staining (using fluorescein, lissamine green or rose Bengal stain), Shirmer's strip testing, Zone-Quick threads, tear film osmolarity, tear break-up-time and tear meniscus height.

The “effective amount” of azithromycin administered to a subject is an amount effective to reduce the clinical signs and/or symptoms of dry eye disease. The present invention is not limited to the use of free base of azithromycin, it also includes the use of pharmaceutically acceptable salts of azithromycin. Pharmaceutically acceptable salts are salts that retain the desired biological activity of azithromycin and do not impart undesired toxicological effects.

The present invention is concerned primarily with the treatment of human subjects, but can also be employed for the treatment of other mammalian subjects, such as dogs and cats, for veterinary purposes.

Azithromycin can be administered to the eyes of a patient by any suitable means, including topical administration and systemic administration.

For topical administration, azithromycin is administered to the ocular surface of a subject, in an amount effective to reduce dry eye symptoms and to improve the tear film. Preferably, azithromycin is administered as a liquid or gel suspension in the form of drops, spray or gel. Alternatively, azithromycin can be applied to the eye via liposomes. Further, azithromycin can be infused into the tear film via a pump-catheter system. Azithromycin can also be contained within a continuous or selective-release device, for example, membranes such as, but not limited to, those employed in the Ocusert™ System (Alza Corp., Palo Alto, Calif.). Azithromycin can also be contained within, carried by, or attached to contact lenses or other compatible controlled release materials, which are placed on the eye. Azithromycin can also be contained within a swab or sponge which can be applied to the ocular surface. Azithromycin can also be contained within a liquid spray which can be applied to the ocular surface. Another embodiment of the present invention involves an injection of azithromycin directly into the lacrimal tissues or onto the eye surface.

The topical solution containing azithromycin can contain a physiologically compatible vehicle, as those skilled in the ophthalmic art can select using conventional criteria. The ophthalmic vehicles include, but are not limited to, saline solution, artificial tears, water polyethers such as polyethylene glycol, polyvinyls such as polyvinyl alcohol and povidone, cellulose derivatives such as methylcellulose and hydroxypropyl methylcellulose, polycarbophil, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

The topical formulation optionally includes a preservative, such as benzalkonium chloride and other inactive ingredients such as EDTA. The pH of the formulation is adjusted by adding any physiologically and ophthamologically acceptable pH adjusting acids, bases or buffers to within the range of about 5 to 7.5; preferably 6 to 7. Examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include citrate/dextrose, sodium bicarbonate, ammonium chloride and mixtures of the aforementioned acids and bases.

The osmotic pressure of the topical formulation of azithromycin is generally from about 200 to about 400 milliosmolar (mOsM), more preferably from 260 to 340 mOsM. The osmotic pressure can be adjusted by using appropriate amounts of physiologically and ophthamologically acceptable ionic or non-ionic agents. Sodium chloride is a preferred ionic agent, and the amount of sodium chloride ranges from about 0.01% to about 1% (w/v), and preferably from about 0.05% to about 0.45% (w/v). Equivalent amounts of one or more salts made up of cations such as potassium, ammonium and the like and anions such as chloride, citrate, ascorbate, borate, phosphate, bicarbonate, sulfate, thiosulfate, bisulfate, sodium bisulfate, ammonium sulfate, and the like can be used in addition to or instead of sodium chloride to achieve osmolality within the above-stated range. Further, non-ionic agents such as mannitol, dextrose, sorbitol, glucose and the like can also be used to adjust the osmolality.

The concentration of azithromycin included in the topical formulation is an amount sufficient to reduce dry eye symptoms and/or improve the tear film. This formulation is preferably an aqueous solution of azithromycin and is in the range of 0.005-3%, preferably 0.01% to 2%, preferably 0.1-2%, more preferably 0.5-1.5%, and most preferably about 1.0% (w/v). “About” as used herein, refers to ±15% of the recited value. The formulation optionally includes a preservative, such as benzalkonium chloride (0.003% w/v) and inactive ingredients: edetate sodium, purified water, sodium chloride, sodium phosphate monobasic, sodium hydroxide, and/or hydrochloric acid to adjust the pH to about 6-8, preferably about 7.

A preferred ophthalmic formulations of azithromycin suitable for the present method are those disclosed in U.S. Pat. Nos. 6,239,113, 6,569,443 and 7,056,893; the formulations of which are incorporated herein by reference. For example, the formulation is an aqueous polymeric suspension comprising water, azithromycin, and 0.1 to 10% of a polymeric suspending agent. The polymeric suspending agent comprises a water-swellable water-insoluble crosslinked carboxy-vinyl polymer. For example, the polymeric suspending agent comprises least 90% acrylic acid monomers and 0.1% to 5% crosslinking agent. AZASITE® (azithromycin ophthalmic solution), which is a 1% sterile aqueous topical ophthalmic solution of azithromycin formulated in DURASITE® (polycarbophil, edetate disodium, sodium chloride), is the most preferred ophthalmic formulation. The preferred ophthalmic formulations are able to keep prolonged high azithromycin concentration on the ocular surface, thus facilitating its penetration into the eye tissues.

The daily topical dose to reduce dry eye symptoms and improve tear film composition can be divided among one or several unit dose administrations. The total daily dose for azithromycin, for example, can range from one drop (about 50 μl), one to four times a day, depending upon the age and condition of the subject. A preferred regimen for azithromycin is one drop of 1.0% (w/v) solution, about 1 to 2 times a day.

In addition to the topical method of administration described above, there are various methods of administering azithromycin systemically. One such method involves an aerosol suspension of respirable particles comprised of azithromycin, which the subject inhales. Azithromycin is absorbed into the bloodstream via the lungs or via nasolacrimal ducts, and subsequently contact the lacrimal glands in a pharmaceutically effective amount. The respirable particles can be liquid or solid, with a particle size sufficiently small to pass through the mouth and larynx upon inhalation; in general, particles ranging from about 1 to 10 microns, but more preferably 1-5 microns, in size are considered respirable.

Liquid pharmaceutical compositions of azithromycin for producing a nasal spray or nasal or eye drops can be prepared by combining azithromycin with a suitable vehicle, such as sterile pyrogen free water or sterile saline by techniques known to those skilled in the art.

Other method of systemic administration of the active compound involves oral administration, in which pharmaceutical compositions containing azithromycin are in the form of tablets, lozenges, aqueous or oily suspensions, dispersible powders or granules, emulsion, hard or soft capsules, or syrups or elixirs. Compositions intended for oral use can be prepared according to any method known to the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain azithromycin in admixture with nontoxic pharmaceutically acceptable excipients, which are suitable for the manufacture of tablets. These excipients can be, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets can be uncoated or they can be coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

A preferred systemic administration is oral administration. In a tablet, a preferred dose contains 1-2000 mg, preferably 50-1000 mg and most preferably 250-500 mg of azithromycin, and is administered once or twice a day. Alternately, an oral syrup or dry syrup such as 1-2 teaspoons of a 1% (w/v) azithromycin suspension can be administered to a subject once or twice a day.

Additional method of systemic administration of azithromycin to the eyes of the subject involves a suppository form of the active compound, such that a therapeutically effective amount of the compound reaches the eyes via systemic absorption and circulation.

Further method of administration of azithromycin involves direct intra-operative instillation of a gel, cream, powder, foam, crystal, liposomes, spray or liquid suspension form of said compound, such that a therapeutically effective amount of the compound reaches the eyes via systemic absorption and circulation.

The invention is illustrated further by the following example, which is not to be construed as limiting the invention to the specific procedures described in it.

### EXAMPLES

### Example 1

### Use of Azithromycin for Reducing Symptoms in Patients With Dry Eye Disease

### Objectives

The objective of this study is to compare the efficacy of study drug, AZASITE® (azithromycin ophthalmic solution) 1%, versus the vehicle (DuraSite®) over a four week treatment period on the symptoms of subjects with dry eye disease.

### Subjects

Subjects are 18 years of age or older, and have a clinical diagnosis of mild to moderate dry eye disease. A total of 100 subjects are enrolled in the study.

### Methods

This is a double-masked study. At Visit 1 (Day 1), all subjects are randomized in 1:1 ratio to receive either (a) AZASITE® or (b) the vehicle DURASITE®), for 30 days. Study drug is administered as one drop in each eye BID for the first 2 days and then QD for the remainder of the study. Study drugs are self-administered by the subjects. The subjects are prohibited in using any ocular or other medications that could confound the results of the assessments during study participation, such as antihistamines, steroids, antibiotics or preserved artificial tears.

Patients return for Visit 2 (Day 14), and Visit 3 (Day 30) and are asked to rate their symptoms including: ocular itching, ocular burning/pain, and foreign body sensation.

### Scores on the Symptoms of Dry Eye Disease

Patients rate the severity of their dry eye symptoms at Visits 1, 2, and 3 according to the following three classifications.

### Eyelid Itching

Do your eyelids feel itchy?
(0) None: My eyelids do not feel itchy.
(1) Mild: Once in a while, my eyelids feel slightly itchy, but I do not have a desire to rub them.
(2) Moderate: Occasionally, my eyelids feel itchy, and I need to rub them.
(3) Severe: It is difficult to relieve the sensation of itchiness even when I rub my eyelids.
(4) Very severe: I have unbearable eyelid itching with an irresistible urge to rub my eyelids.

### Foreign Body Sensation/Sandiness, Grittiness

Do you feel like there's something sandy or gritty in your eye?
(0) None: My eyes do not feel sandy or gritty.
(1) Mild: I am aware of the surface of my eyes once in a while.
(2) Moderate: My eyes feel like there is something small in them occasionally.
(3) Severe: My eyes feel like there is something large or gritty in them.
(4) Very severe: I am unable to open my eyes due to feeling of a foreign body in my eyes.

### Ocular Burning or Pain

Are your eyes burning or painful?
(0) None: My eyes do not burn or ache.
(1) Mild: I am aware of the surface of my eyes; they mildly burn or ache.
(2) Moderate: I feel my eyes are burning, but still tolerable
(3) Severe: My eyes feel throbbing or fiery due to burning/pain.
(4) Very severe: I am unable to open my eyes due to burning/pain

### Results

The mean scores for individual symptoms for each group (AZASITE® and DURASITE®), are compared for Visits 2-3 to baseline (Visit 1). A statistically significant difference (p<0.05) is observed in favor of the AZASITE® treatment group for at least one of the Visits.

### CONCLUSIONS

The above results indicate that AZASITE® improves the symptoms of dry eye disease significantly greater than the vehicle, DURASITE®.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

What is claimed is:

1. A method for treating dry eye diseases, comprising the steps of:
(i) identifying a subject suffering from dry eye disease; and
(ii) administering to the eyes of the subject an aqueous ophthalmic solution comprising: (a) an active pharmaceutical ingredient consisting essentially of azithromycin, and (b) a physiologically compatible ophthalmic vehicle;
wherein said dry eye disease is caused by one or more of keratoconjunctivitis sicca, age-related dry eye, Stevens-Johnson syndrome, Sjogren's syndrome, ocular cicatrical pemphigoid, corneal injury, infection, Riley-Day syndrome, congenital alacrima, nutritional disorders or deficiencies, pharmacologic side effects, contact lens intolerance, eye stress resulting in glandular and tissue destruction, autoimmune disorders, immuno-deficient disorders, comatose patients who are unable to blink, or environmental exposure to smog, smoke, excessively dry air, or airborne particulates.

2. The method according to claim 1, wherein said dry eye disease is caused by keratoconjunctivitis sicca, age-related dry eye, Stevens-Johnson syndrome, Sjogren's syndrome, ocular cicatrical pemphigoid, corneal injury, Riley-Day syndrome, or congenital alacrima.

3. The method according to claim 1, wherein said dry eye disease is caused by nutritional disorders or deficiencies, contact lens intolerance, autoimmune disorders, immuno-deficient disorders, comatose patients who are unable to blink, or environmental exposure to smog, smoke, excessively dry air, or airborne particulates,

4. The method according to claim 1, whereby one or more dry eye symptoms are reduced or alleviated in the subject, wherein the dry eye symptoms are selected from the group consisting of dryness, burning, ocular itching, photophobia, foreign body sensation, and grittiness.

5. The method according to claim 1, wherein said aqueous ophthalmic solution comprises 0.5-1.5% (w/v) azithromycin, polycarbophil, edetate disodium, and sodium chloride.

6. The method according to claim 5, wherein the amount of the azithromycin is about 1% (w/v).

7. The method according to claim 1, wherein said physiologically compatible ophthalmic vehicle comprises artificial tears.

8. A method for treating dry eye diseases, comprising the steps of:
(i) identifying a subject suffering from dry eye disease and not suffering from blepharitis; and
(ii) administering to the subject an aqueous ophthalmic solution comprising: (a) an active ingredient consisting essentially of azithromycin, and (b) a physiologically compatible ophthalmic vehicle.

9. The method according to claim 8, whereby one or more dry eye symptoms are reduced or alleviated in the subject, wherein the dry eye symptoms are selected from the group consisting of dryness, burning, ocular itching, photophobia, foreign body sensation, and grittiness.

10. The method according to claim 8, wherein said aqueous ophthalmic solution comprises 0.5-1.5% (w/v) azithromycin, polycarbophil, edetate disodium, and sodium chloride.

11. The method according to claim 10, wherein the amount of azithromycin is about 1% (w/v).

12. A method for treating dry eye diseases, comprising the steps of:
(i) identifying a subject suffering from dry eye disease; and
(ii) administering to the eyes of the subject an aqueous ophthalmic solution comprising: (a) active pharmaceutical ingredients consisting essentially of azithromycin and a secretegogue, and (b) a physiologically compatible ophthalmic vehicle.

13. The method according to claim 12, wherein said secretegogue is diquafosol, 15-HETE, or rebamipide.

14. The method according to claim 12, whereby one or more dry eye symptoms are reduced or alleviated in the subject, wherein the dry eye symptoms are selected from the group consisting of dryness, burning, ocular itching, photophobia, foreign body sensation, and grittiness.

15. A method for treating dry eye diseases in a subject prior to an ophthalmic surgical procedure, comprising the steps of:
identifying a subject having dry eye disease or being at risk of developing post surgery dry eye disease, and
administering to the subject an effective amount of azithromycin prior to the ophthalmic surgical procedure.

16. The method according to claim 15, wherein said ophthalmic surgical procedure is refractive or cataract surgery.

17. The method according to claim 15, whereby one or more dry eye symptoms are reduced or alleviated in the subject, wherein the dry eye symptoms are selected from the group consisting of dryness, burning, ocular itching, photophobia, foreign body sensation, and grittiness.

18. A method for treating dry eye diseases in a subject following an ophthalmic surgical procedure, comprising the steps of:
identifying a subject suffering from dry eye disease following an ophthalmic surgical procedure, and
administering to the subject azithromycin.

19. The method according to claim 18, wherein said ophthalmic surgical procedure is refractive or cataract surgery.

20. The method according to claim 18, whereby one or more dry eye symptoms are reduced or alleviated in the subject, wherein the dry eye symptoms are selected from the group consisting of dryness, burning, ocular itching, photophobia, foreign body sensation, and grittiness.
